(19) United States
(12) Patent Application Publication
(10) Pub. No.: US 2018/0014750 A1
(43) Pub. Date: Jan. 18, 2018

(54) LIGHT BASED LOCATION AND IDENTIFICATION OF IMPLANTED MEDICAL DEVICES

(71) Applicant: MEDICAL COMPONENTS, INC., Harleysville, PA (US)

(72) Inventor: Premkumar Babu, Allentown, PA (US)

(21) Appl. No.: 15/717,257

(22) Filed: Sep. 27, 2017

Related U.S. Application Data
(62) Division of application No. 14/613,940, filed on Feb. 4, 2015.
(60) Provisional application No. 61/935,527, filed on Feb. 4, 2014.

(51) Int. Cl. A61B 5/06; A61M 39/02; A61B 5/00; A61M 39/04

(52) U.S. Cl. CPC A61B 5/064; A61B 5/743; A61B 90/37; A61B 90/94; A61B 5/0075; A61M 39/0208; A61B 2560/0425; A61B 2090/365; A61B 2090/373; A61B 2562/0238; A61B 2090/366; A61M 39/04; A61M 2039/0205; A61M 2039/0238

(57) ABSTRACT

A method of determining information regarding an implanted medical device. The method includes scanning an infrared or near-infrared laser over a target area wherein a medical device having at least one light affective area defined thereon is implanted; sensing reflected light from the scanned area; processing the reflected light and creating an image which indicates the light affective area based on a difference in the sensed light; and displaying the created image. A system for determining information regarding an implanted medical device is also provided.

### CROSS REFERENCE TO RELATED APPLICATIONS

This application is a divisional of U.S. patent application Ser. No. 14/613,940, filed on Feb. 4, 2015, which claims the benefit of U.S. Provisional application Ser. No. 61/935,527, filed Feb. 4, 2014; the entireties of which are incorporated herein by reference.

### FIELD OF THE INVENTION

This invention relates generally to medical devices, and more particularly to location and identification of implanted medical devices utilizing a light based system.

### BACKGROUND OF THE INVENTION

Implantable medical devices are devices which are implanted subcutaneously and provide a biological function for the patient. Such implantable medical devices include, for example, screws, pins, plates, pods, artificial joints, coronary stents, cardioverter defibrillators, heart pacemakers, IUDs (Intra-Uterine Devices), catheters and venous access ports.

After implantation, and sometimes during the implantation procedure, it is necessary to locate the position and/or orientation of the implanted medical device and also to confirm or determine the attributes of the medical device. In many situations, the subcutaneous device is not visible, and therefore, other techniques must be utilized to determine the location, orientation or attributes of the medical device. Exemplary techniques include palpation, i.e. feeling for the device, and x-ray imaging.

While these techniques have met some needs, each also has drawbacks. In one exemplary application, namely a venous access port, palpation requires the practitioner to feel for the subcutaneous port within a fat layer of the patient. In some instances, the practitioner may feel some other hardened object and mistakenly identify it as the port. Additionally, even if the port is located, the practitioner may have difficulty specifically locating the septum portion of the port and several needle sticks in the general area may be necessary. Alternatively, the practitioner may correctly locate the port, however, if the port has rotated such that the septum is not partially or completely available, the practitioner may not be able to determine such until after several needle sticks. While x-ray imaging provides a more precise view of the subcutaneous port, it may have its own drawbacks. Firstly, x-ray imagining typically requires the patient to be transported to a specific x-ray imagining location, which delays the procedure and requires additional resources. Additionally, each x-ray procedure exposes the patient to additional radiation, which generally is desired to be kept to a minimum.

It is desired to provide a system and method that provides a medical practitioner with capability to discern one or more properties of an implantable medical device.

### SUMMARY OF THE INVENTION

In at least one embodiment, the present invention provides a method of determining information regarding an implanted medical device. The method includes scanning an infrared or near-infrared light over a target area wherein a medical device having at least one light affective area defined thereon is implanted. By light affective area, it is meant that the area affects light more by having a greater absorption or reflection effect on infrared or near-infrared light then surrounding areas. The method further includes receiving reflected light from the scanned area or sensing the absorbed light; processing the reflected or absorbed light and creating an image which indicates the light affective area based on a difference in the received reflected or absorbed light; and displaying the created image.

In at least one embodiment, the step of displaying the created image includes projecting the created image as visible light on the target area.

In at least one embodiment, the information to be determined includes one of location, orientation or attribute of the medical device.

In at least one embodiment, the invention provides a system for determining information regarding an implanted medical device. The system includes a medical device having at least one light affective area defined thereon; and a light sensing device. The light sensing device is configured to scan a target area with an infrared or near-infrared light; receive reflected light or sense absorbed light from the target area; and create an image which indicates the light affective area based on a difference in the received reflective or absorbed light.

In at least one embodiment, the invention provides a venous access port including a body defining an internal reservoir in communication with a discharge port; a septum positioned above the internal reservoir; and at least one light affective area configured to identify the location of the septum, the at least one light affective area absorbs or reflects infrared or near-infrared light differently than the remainder of the body. In one embodiment, the at least one light affective area is defined about the septum. In yet another embodiment, the at least one light affective area is defined by the septum.

### BRIEF DESCRIPTION OF THE DRAWINGS

The accompanying drawings, which are incorporated herein and constitute part of this specification, illustrate the presently preferred embodiments of the invention, and, together with the general description given above and the detailed description given below, serve to explain the features of the invention. In the drawings:

FIG. 1 is a perspective view illustrating use of a light scanning device to visualize an implanted exemplary venous access port in accordance with an exemplary embodiment of the invention.

FIG. 2 is a perspective view illustrating use of a light scanning device to visualize an implanted exemplary catheter in accordance with an exemplary embodiment of the invention.

FIG. 3 is a perspective view of one embodiment of a venous access port useable in accordance with the present invention.

FIG. 4 is a top plan view of the port of FIG. 3.

FIG. 5 is a bottom plan view of the port of FIG. 3.

FIG. 6 is a cross-sectional view along the line 6-6 in FIG. 4.

FIG. 7 is a cross-sectional view along the line 7-7 in FIG. 4.

FIG. 8 is a top plan view similar to FIG. 4 illustrating an alternatively marked port.

FIG. 9 is a top plan view of an alternative venous access port useable in accordance with the present invention.

FIG. 10 is a cross-sectional view along the line 10-10 in FIG. 9.

FIG. 11 is a bottom plan view of the port of FIG. 9.

FIG. 12 is an illustrative image of the light detected by the light scanning device as applied to the port of FIG. 3.

FIG. 13 is an illustrative projected image from the light scanning device based upon the light detected as illustrated in FIG. 12.

FIG. 14 is an alternative projected image from the light scanning device based upon light detected from the port of FIG. 8.

FIG. 15 is an alternative projected image from the light scanning device based upon light detected from an alternative venous access port.

FIG. 16 is an alternative projected image from the light scanning device based upon light detected from the port of FIG. 9.

FIGS. 17 and 18 are illustrative projected images from the light scanning device based upon light detected from the catheter of FIG. 2.

### DETAILED DESCRIPTION OF THE INVENTION

In the drawings, like numerals indicate like elements throughout. Certain terminology is used herein for convenience only and is not to be taken as a limitation on the present invention. The following describes preferred embodiments of the present invention. However, it should be understood, based on this disclosure, that the invention is not limited by the preferred embodiments described herein.

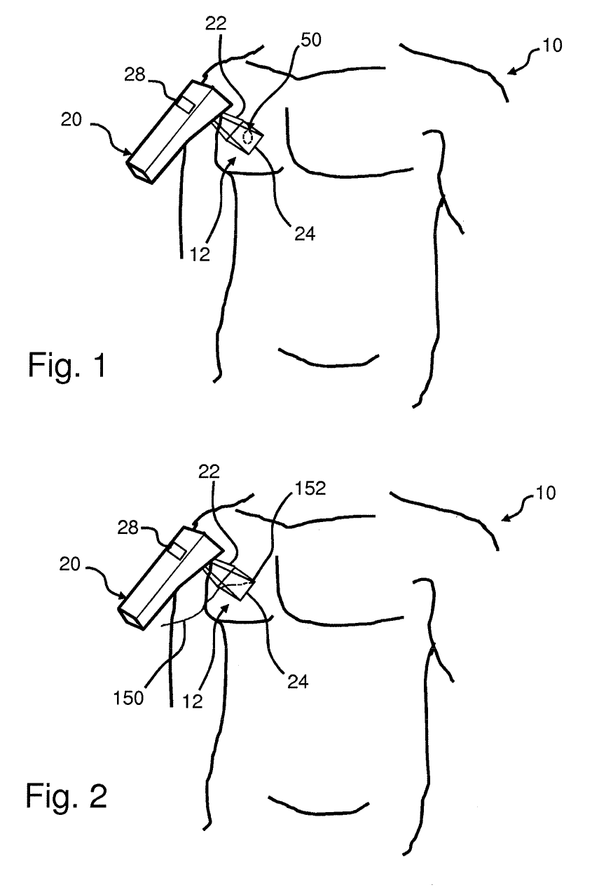

Referring to FIGS. 1 and 2, an exemplary light scanning device 20 is illustrated in use to detect an implanted medical device, i.e. a venous access port 50 in FIG. 1 and a catheter 150 in FIG. 2. The exemplary light scanning device 20 uses a two-axis optical scanner to sweep an infrared (IR) or near-infrared (NIR) laser 22 over a target area 12 of a patient 10. The sweeping laser 22 defines a two-dimensional field of view 24. As explained in more detail hereinafter, the implanted medical device 50, 150 is formed with at least one light affective area which is configured to affect the IR or NIR light differently compared to the surrounding tissue and the remaining components of the medical device 50, 150. For example, the medical device 50, 150 may have an area configured to absorb the IR or NIR light, such that less of the light is reflected in that area or an area configured to reflect the IR or NIR light, such that more of the light is reflected in the area compared to surrounding areas.

At the same time, the light scanning device 20 is configured to receive and record the reflected light from the field of view 24 using photodiodes or the like tuned to the wavelength of the laser 22. The light sensing device 20 includes a processor (not shown) which receives signals corresponding to the received reflected light and utilizes digital signal processing or the like to create an image of the sensed field of view. The light sensing device 20 may include a display 28 upon which the image is displayed. Additionally, the light sensing device 20 is further configured to reproject the created image onto the skin using a visible laser. In an exemplary embodiment, the IR or NIR laser 22 has a wavelength of approximately 785 nm and the visible laser has a wavelength of approximately 642 nm. Aligned with the data acquired in the infrared range, the projected image provides the practitioner with direct and immediate feedback on the location and orientation of the implanted medical device 50, 150.

The light scanning device 20 may have various internal components for generating and detecting the IR or NIR light and for generating the projected image using visible light. Additionally, the light scanning device 20 may be a stationary device or a portable device. Various systems for carrying out such light sensing and image generation are set forth in U.S. Pat. Nos. 8,073,531; 8,255,040; 8,295,904; 8,328,368; 8,380,291; 8,391,960; 8,463,364; 8,478,386; 8,489,178; and 8,594,770, each of which is incorporated herein by reference.

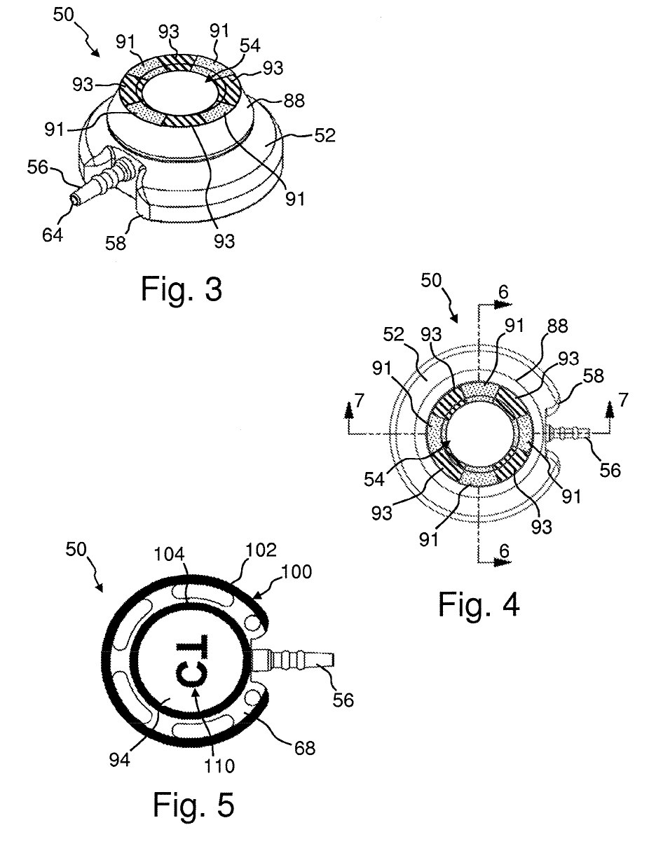

As set forth above, the medical devices 50, 150 are preferably formed with at least one light affective area which is configured to interact differently with the IR or NIR light compared to the surrounding tissue and the remaining components of the medical device 50, 150. Exemplary venous access ports 50 incorporating such a light affective area will be described with reference to FIGS. 3-11.

Referring to FIGS. 3-7, a first exemplary venous access port 50 will be described. The port 50 generally has a structure similar to the port structure disclosed in U.S. Pat. No. 8,257,325, the contents of which are incorporated herein by reference. Generally, the venous access port 50 includes a housing 52 and a septum 54, with a discharge port 56 extending from a distal end 58 of the port assembly 50 to be attached securely and sealingly to the proximal end of a catheter (not shown). A passageway 60 extends from the interior reservoir 62 to the distal tip opening 64 of discharge port 56.

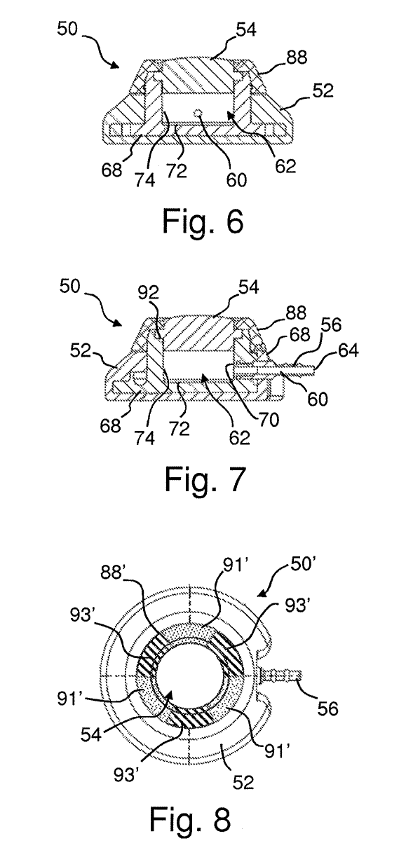

With reference now to FIGS. 6 and 7, the interior of the port assembly 50 is shown to provide an interior reservoir 62. The housing 52 is shown to include a housing base 68 of needle-impenetrable material that includes a well 70 having a bottom floor 72 and side walls 74 that define the interior reservoir 62 beneath septum 54. A skirt 82 is overmolded about housing base 68 and may be of silicone elastomer or other biocompatible material. A cap 88 is also secured to housing base 68 to in turn secure the septum 54 in position in the port assembly 50. The housing base 68 includes a septum seat 92 extending into the top of well 70, into which a flange of the septum will be seated.

Referring to FIGS. 3 and 4, the cap 88 defines a series of light affective areas 91, 93 annularly about the septum 54. With this illustrative port 50, four darker absorptive areas 91 alternate with four lighter absorptive areas 93. The darker absorptive areas 91 are configured to absorb more of the IR or NIR light 22 such that less reflected light will be received for these areas while the lighter absorptive areas 93 are configured to absorb less of the IR or NIR laser 22 light such that more reflected light will be received for these areas.

The light affective areas 91, 93 may be defined utilizing various techniques. As examples, the light affective areas 91, 93 may be defined to have a color which is absorptively or reflectively distinct from the other areas and other components. Alternatively, materials having different IR or NIR absorptive or reflective qualities may be selected. For example, all or portions of the cap 88 may be made from absorptively or reflectively distinct material. In another embodiment, the septum 54 itself may be manufactured from an absorptively or reflectively distinct material such that the septum defines the light affective area and appears as a distinctly lighted area of the reprojected image 26. As another exemplary alternative, the light affective areas 91, 93 may be defined by coating the areas with materials which are absorptively or reflectively distinct, e.g. materials having different fluorescence. As yet another exemplary alternative, the light affective areas 91, 93 may have varying configurations, e.g. different concavities or convexities, which cause absorptively or reflectively distinct areas. Other mechanisms, for example, light producing LEDs or light reflective metals, coatings or the like, may be positioned in the light affective areas 91, 93. The invention is not limited to these exemplary mechanisms for achieving absorptively or reflectively distinct areas and other mechanisms may be utilized.

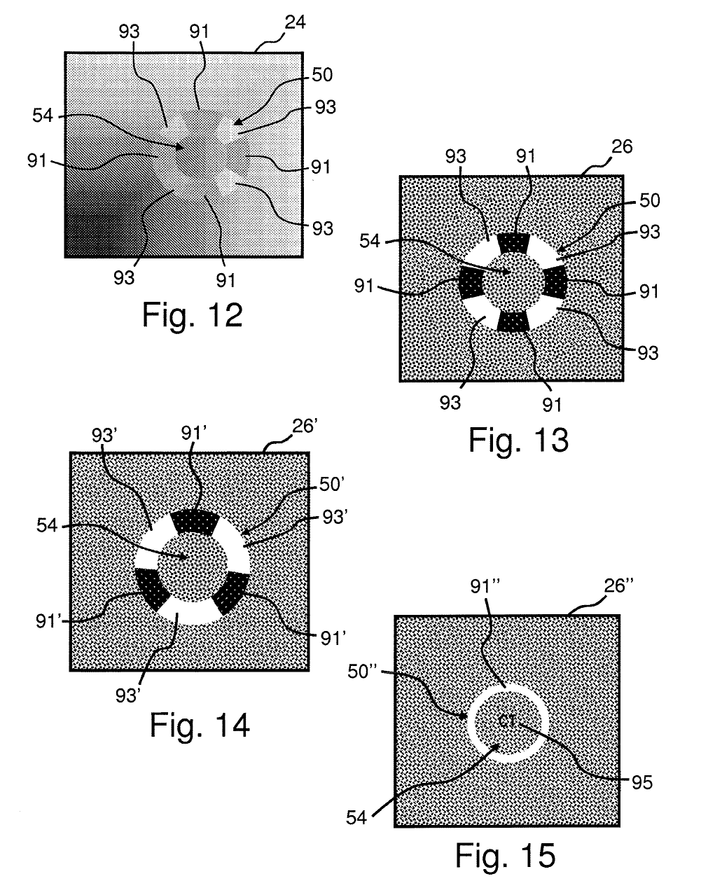

The received reflected light for this embodiment of the port 50 is illustrated in FIG. 12, with the areas corresponding to the darker absorptive areas 91 appearing darker than the areas corresponding to the lighter absorptive areas 93. While this embodiment is described with the light affective areas 91, 93 as absorptive areas, it is recognized that one or both of the areas may instead be reflective areas. As illustrated, each of the light affective areas 91, 93 is distinct from the surrounding area and the remainder of the port 50, including the septum 54. In this way, the septum 54 is clearly identifiable as it is encircled by the light affective areas 91 and 93.

FIG. 13 illustrates an exemplary reprojected visible image 26 based on the light received as illustrated in FIG. 12. The light sensing device 20 is preferably configured to process the received signals such that the reprojected visible image 26 corresponds to the received light, but is sharper and clearer at the contrasting areas. As seen, the reprojected image 26 will allow the practitioner to clearly identify the location of the port 50, but more specifically the target septum 54. The practitioner can easily insert a needle into the septum 54 without the need to guess its exact location as may be required using the palpation technique. Additionally, the sensed light and projected image 26 allow the practitioner to easily determine the orientation of the port 50. If the port 50 began to turn in the patient, the practitioner would see such change in orientation and corrective measures may be initiated more promptly than with prior techniques.

The light affective areas 91, 93 may also be utilized to provide attributes of the medical device. For example, the port 50 of FIGS. 3-7 is a power injectable port and the pattern of the light affective areas 91, 93 (e.g. four alternating areas) may be configured to convey that the port 50 is power injectable. Contrast this to the port 50′ illustrated in FIG. 8 which is generally the same as the port 50, but which is not power injectable. The cap 88′ includes a series of light affective areas 91′ and 93′ about the septum 54, however, only three of each light affective area 91′, 93′ are provided. A standard may be set such that the alternating pattern of three light affective areas 91′, 93′ corresponds to a non-power injectable port 50′. Comparing the reprojected visible image 26′ of the port 50′ in FIG. 14 with the image 26 of the port 50 in FIG. 13, it would be clear to the practitioner which port is power injectable and which is not. As an alternative, the non-power ports may be made without any absorptive/reflective pattern that by its very absence would be clear to the practitioner that the port is non-power injectable.

As illustrated in FIG. 5, the port 50 may still include radiopaque markings 100 to designate the port 50 as power injectable. A larger outer circle 102 is seen provided on the outermost periphery of bottom base surface 94 and a smaller inner circle 104 is seen provided more centrally. The outer and inner circles or rings 102, 104 circumscribe radiopaque indicia 110. Other radiopaque markings 100 distinct from the elements shown may also be utilized. Inclusion of the radiopaque markings 100 in addition to the light affective areas 91, 93 allows the attributes of the port 50 to be identified in the event a light sensing device 20 is not available or in the event that the patient is undergoing an x-ray anyway.

Referring to FIG. 15, the location/orientation function may be distinct from the attribute identification function. For example, the reprojected image 26″ represents a power injectable port 50″ in which a single light affective area 91″ encircles the septum 54 to provide an indication of the location and orientation of the septum 54. A second light affective area 95 in the form of an alphanumeric indicia, i.e. “CT”, is defined in the middle of the septum 54 and is visible in the reprojected image 26″. Other standards may be utilized be to convey attribute information. Also, additional attribute information other than the power injectability may be incorporated into the information represented by the light affective areas.

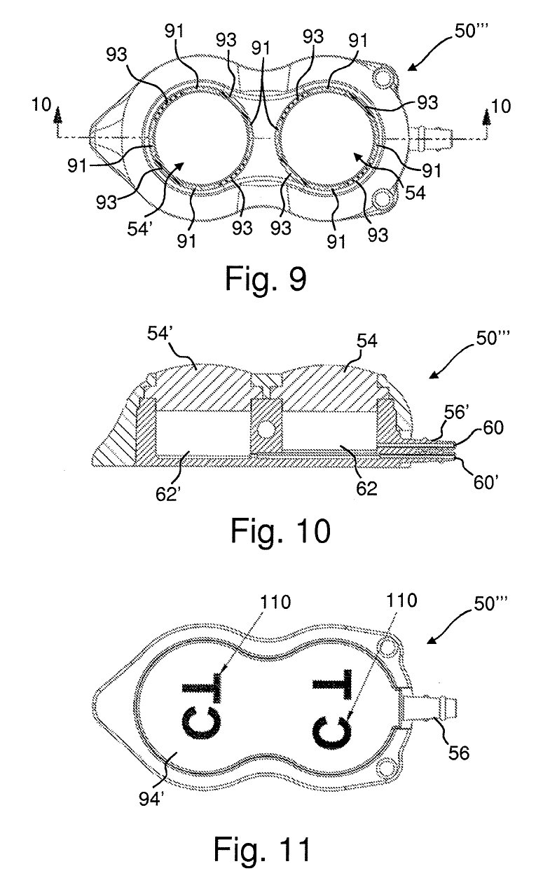

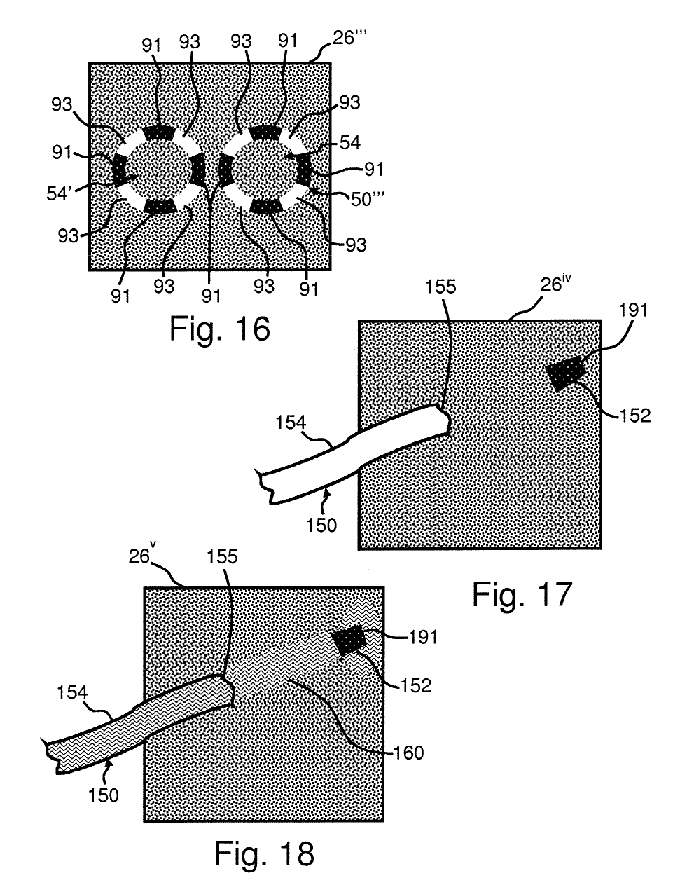

Referring to FIGS. 9-11 and 16, another exemplary port 50′″ will be described. The port 50′″ is similar to the previous embodiments, but provides a dual port with a pair of septums 54, 54′ positioned over respective reservoirs 62, 62′. A respective passageway 60, 60′ extends from each interior reservoir 62, 62′ to the distal tip openings of discharge port 56. As illustrated in FIG. 9, a series of light affective areas 91, 92 may be provided about each septum 54, 54′ such that the two distinct target areas will be visible in the reprojected image 26″, as illustrated in FIG. 16.

Referring to FIGS. 2 and 17-18, use of the light sensing device with an alternative implantable medical device, namely, a catheter 150, will be described. In the illustrated embodiment, a light affective area 191 is defined at the tip 152 of the catheter tube 154. The light affective area 191 is similar to the light affective areas described above. While the illustrated embodiment includes light affective area 191 only at the tip 152, it may be provided over a larger area, at spaced intervals or any other desired configuration. As illustrated in FIG. 17, the reprojected image 26.sup.iv will show the position of the tip 152 as the catheter body 154 is advanced through an incision 155 and under the skin of the patient. Additionally or alternatively, the configuration of the light affective area 191 may be selected such that it is absorptively or reflectively distinct from a fluid 160 intended to be passed through the catheter 150 such that both the tip 152 and the presence of the fluid 160 in the catheter are visible on the reprojected image 26.sup.v, as illustrated in FIG. 18.

While the present invention has been described with respect to various venous access ports and catheters, the invention is not limited to such and other medical devices may be formed with light affective areas and a light scanning device may be utilized to determine location, orientation, attributes and other information about the subcutaneously implanted medical devices.

These and other advantages of the present invention will be apparent to those skilled in the art from the foregoing specification. Accordingly, it will be recognized by those skilled in the art that changes or modifications may be made to the above-described embodiments without departing from the broad inventive concepts of the invention. It should therefore be understood that this invention is not limited to the particular embodiments described herein, but is intended to include all changes and modifications that are within the scope and spirit of the invention as defined in the claims.

What is claimed:

1. A method of determining information regarding an implanted medical device, the method comprising:
scanning an infrared or near-infrared laser over a target area wherein a medical device having at least one light affective area defined thereon is implanted;
sensing reflected light from the scanned area;
processing the reflected light signal and creating an image which indicates the light affective area based on a difference in the received reflected light signal; and
displaying the created image.

2. The method of claim 1, wherein the step of displaying the created image includes projecting the created image as visible light on the target area.

3. The method of claim 2, wherein the visible light is a visible laser with a different wavelength than the infrared or near-infrared laser.

4. The method of claim 2, wherein the projecting of the created image as visible light on the target area is done in near real time.

5. The method of claim 1, wherein the step of displaying the created image includes displaying the created image on a display of a scanning device which is used for generating the infrared or near-infrared laser.

6. The method of claim 1, wherein the information to be determined includes one of location, orientation or attribute of the medical device.

7. The method of claim 6, wherein a pattern of the light affective area is indicative of an attribute of the medical device.

8. The method of claim 7, wherein the medical device is a venous access port and the pattern of the light affective area is indicative of whether the venous access port is power-injectable or not.

9. The method of claim 1, wherein the medical device is a catheter and the at least one light affective area is provided at least on a distal end of the catheter.
